(19) United States
(12) Patent Application Publication
(10) Pub. No.: US 2009/0264479 A1
(43) Pub. Date: Oct. 22, 2009

(54) Papain Family Cysteine Protease Inhibitors for the Treatment of Parasitic Diseases

(76) Inventors: Cameron Black, Baie d'Urfe (CA); Christophe Mellon, L'lle-Bizard (CA); Deborah Anne Nicoll-Griffith, Baie d'Urfe (CA); Renata Oballa, Kirkland (CA)

Correspondence Address: MERCK AND CO., INC, P O BOX 2000, RAHWAY, NJ, 07065-0907, US

(21) Appl. No.: 11/989,437

(22) PCT Filed: Jul. 24, 2006
(86) PCT No.: PCT/CA2006/001216

§ 371 Date: Jan. 24, 2008

Related U.S. Application Data
(60) Provisional application No. 60/702,455, filed on Jul. 26, 2005.

(51) Int. Cl. A61K 31/4402; A61K 31/165; A61P 33/00

(52) U.S. Cl. 514/357; 514/620

(57) ABSTRACT

Several parasites responsible for mammalian diseases are dependent on cysteine protease for various life-cycle functions. Inhibition of these proteases can be useful in the treatment of these parasitic diseases, including toxoplasmosis, malaria, African trypanosomiasis, Chagas disease, leishmaniasis or schistosomiasis.

### BACKGROUND OF THE INVENTION

Several parasites responsible for mammalian diseases are dependent on cysteine protease for various life-cycle functions. Inhibition of these proteases can be useful in the treatment of these parasitic diseases, see Lecaille, F., et al, Chem. Rev., 102, 4459-4488, 2002.

Cruzipain is a cysteine protease enzyme present in Trypanosoma cruzi and is thought to play an important role in all stages of the parasite's life cycle. The enzyme is highly expressed in the epimastigote stage where it is primarily a lysosomal enzyme and may be involved in protein digestion during differentiation to the infective metacyclic trypomastigote stage. Identification of cruzipain in the membrane of the trypomastigote implicates this enzyme in the penetration of the parasite into the host cell. Cruzipain is also found in the membranes of the amastigote form of the parasite, see Cazzulo, J. J., et al, Current Pharmaceutical Design, 7, 1143-1156, 2001. Cruzipain efficiently degrades human IgG, which may play a protective role for the parasite by preventing antigen presentation and thus reducing the host immune response. Based on these observations, it has been proposed that cruzipain is a valid drug target for chemotherapy of Chagas disease. Cruzipain has been reported to exist in at least two polymorphic sequences, known as cruzipain 1 and cruzipain 2, both of which may be involved in the viability of Trypanosoma cruzi (Lima, et al, Molecular & Parasitology 114, 41-52, 2001).

A similar role for the cysteine protease trypanopain-Tb has been proposed in the life-cycle of Trypanosoma brucei, the parasite responsible for African trypanosomaisis, or sleeping sickness.

A similar parasite, T. congolense, is responsible for the bovine disease trypanosomiasis. Congopain is the analogous cysteine protease to cruzipain in this parasite.

Falcipain is an important cysteine protease in Plasmodium falicparum. This enzyme is reported to be important in the degradation of host hemoglobin in parasite food vacuoles. The processing of hemoglobin is essential to the growth of the parasite, thus an inhibitor of falcipain should be useful as a treatment for malaria.

Two cysteine proteases, SmCL1 and SmCL2, are present in the human blood fluke Schistosoma mansoni. SmCL1 may play a role in the degradation of host hemoglobin, while SmCL2 may be important to the reproductive system of the parasite (Brady, C. P., et al, Archives of Biochemistry and Biophysics, 380, 46-55, 2000). Inhibition of one or both of these proteases may provide an effective treatment for human schistosomiasis.

LmajcatB and CP2.8ΔCTE are important cysteine proteases of the parasitic protazoa Leishmania major and Leishmania mexicanus respectively, see Alves, L. C., et al, Eur. J. Biochem, 268, 1206-1212, 2001. Inhibition of these enzymes may provide a useful treatment for leishmaniasis.

The present invention relates to compounds that are capable of treating and preventing mammalian parasitic diseases in which the parasite utilizes a critical cysteine protease from the papain family.

### SUMMARY OF THE INVENTION

The present invention relates to a method of treating a parasitic disease with a papain family cysteine protease inhibitor. Examples or parasitic diseases include toxoplasmosis, malaria, African trypanosomiasis, Chagas disease, leishmaniasis or schistosomiasis.

One embodiment of the papain family cysteine protease inhibitors of present invention is illustrated by a compound of Formula I, and the pharmaceutically acceptable salts, stereoisomers and N-oxide derivatives thereof:

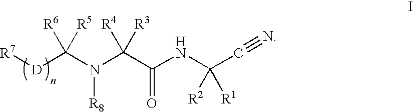

### DETAILED DESCRIPTION OF THE INVENTION

The present invention relates to a method of treating a parasitic disease with a papain family cysteine protease inhibitor. Examples or parasitic diseases include toxoplasmosis, malaria, African trypanosomiasis, Chagas disease, leishmaniasis or schistosomiasis. The present invention also relates to a method of preventing a parasitic disease with a papain family cysteine protease inhibitor. Examples or parasitic diseases include toxoplasmosis, malaria, African trypanosomiasis, Chagas disease, leishmaniasis or schistosomiasis.

One embodiment of the papain family cysteine protease inhibitors of present invention is illustrated by a compound of the following formula, and the pharmaceutically acceptable salts, stereoisomers and N-oxide derivatives thereof:

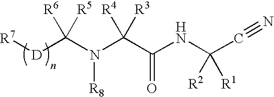

wHerein R¹ is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
R² is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, C₃₋₆ cycloalkyl, halo, hydroxyalkyl, hydroxy, alkoxy or keto; or R¹ and R² can be taken together with the carbon atom to which they are attached to form a C₃₋₈ cycloalkyl ring or heterocyclyl ring wherein said cycloalkyl and heterocycl rings are optionally substituted with one or two substituents independently selected from C₁₋₆ alkyl, hydroxyalkyl, haloalkyl, aryl, heteroaryl, heterocyclyl or halo;
R³ is hydrogen, C₁₋₆ alkyl or C₂₋₆ alkenyl wherein said alkyl and alkenyl groups are optionally substituted with C₃₋₆ cycloalkyl, aryl or halo;
R⁴ is hydrogen, C₁₋₆ alkyl or C₂₋₆ alkenyl wherein said alkyl and alkenyl groups are optionally substituted with C₃₋₆ cycloalkyl, aryl or halo;
or R³ and R⁴ can be taken together with the carbon atom to which they are attached to form a C₃₋₈ cycloalkyl ring, C₅₋₈ cycloalkenyl ring, or five to seven membered heterocyclyl ring wherein said cycloalkyl, cycloalkenyl and heterocyclyl rings are optionally substituted with C₁₋₆ alkyl, halo, hydroxyalkyl, hydroxy, alkoxy or keto;
R⁵ is hydrogen or C₁₋₆ haloalkyl;
R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with halo, C₁₋₆ alkyl, C₁₋₆ haloalkyl, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —N(R¹⁰)(R¹¹), or cyano;
D is C₁₋₃ alkyl, C₂₋₃ alkenyl, C₂₋₃ alkenyl, aryl, heteroaryl, C₃₋₈ cycloalkyl or heterocyclyl wherein said aryl, heteroaryl, cycloalkyl and heterocyclyl groups, which may be monocyclic or bicyclic, are optionally substituted on either the carbon or the heteroatom with one to five substituents selected from C₁₋₆ alkyl, halo or keto;
R⁷ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹⁰, —C(O)OSi[CH(CH₃)₂]₃, —OR¹⁰, —C(O)R¹⁰, —R¹⁰C(O)R⁹, —C(O)R⁹, —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², —SO(R¹²), —SO₂R⁹, —SO₂N(R^{c})(R^{d}), —SO₂CH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)(R⁹), R¹⁰S—, —C(R^{a})(R^{b})NR^{a}C(R^{a})(R^{b})(R⁹), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a}) or C(R^{a})(R^{b})C(O)N(R^{a})(R^{b}); wherein said alkyl, alkenyl, alkynyl, alkoxy, aryl, heteroaryl, cycloalkyl and heterocyclyl groups are optionally substituted on either the carbon or the heteroatom with one to five substituents independently selected from C₁₋₆ alkyl, halo, keto, cyano, haloalkyl, hydroxyalkyl, —OR⁹, —NO₂, —NH₂, —NHS(O)₂R⁸, —R⁹SO₂R¹², —SO₂R¹², —SO(R¹²), —SO₂N(R^{c})(R^{d}), —SO₂N(R¹⁰)C(O)(R¹²), —C(R¹⁰)(R¹¹)N(R¹⁰)(R¹¹), —C(R¹⁰)(R¹¹)OH, —COOH, —C(R^{a})(R^{b})C(O)N(R^{a})(R^{b}), —N(R¹⁰)C(R¹⁰)(R¹¹)(R⁹), —NH(CH₂)₂OH, —NHC(O)OR¹⁰, —Si(CH₃)₃, heterocycyl, aryl or heteroaryl;
R⁸ is hydrogen or C₁₋₆ alkyl;
or R⁴ and R⁸ or can be taken together with any of the atoms to which they may be attached or are between them to form a 4-10 membered heterocyclyl ring system wherein said ring system, which may be monocyclic or bicyclic, is optionally substituted with C₁₋₆ alkyl, halo, hydroxyalkyl, hydroxy, keto, —OR¹⁰, —SR¹⁰ or —N(R¹⁰)₂;
R⁹ is hydrogen, aryl, aryl(C₁₋₄) alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with halo or alkoxy;
R¹⁰ is hydrogen or C₁₋₆ alkyl;
R¹¹ is hydrogen or C₁₋₆ alkyl;
R¹² is hydrogen or C₁₋₆ alkyl which is optionally substituted with halo, alkoxy, cyano, —NR¹⁰ or —SR¹⁰;
R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl or heterocyclyl, wherein said alkyl, aryl, heteroaryl, C₃₋₈ cycloalkyl and heterocyclyl are optionally substituted on either the carbon or the heteroatom with C₁₋₆ alkyl or halo;
R^{b} is hydrogen, C₁₋₆ alkyl, (C₁₋₆ alkyl)aryl, (C₁₋₆ alkyl)hydroxyl, alkoxyl, hydroxyl, halo, aryl, heteroaryl, C₃₋₈ cycloalkyl or heterocyclyl, wherein said alkyl, aryl, heteroaryl, C₃₋₈ cycloalkyl and heterocyclyl are optionally substituted on either the carbon or the heteroatom with C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with C₁₋₆ alkyl and halo;
n is an integer from zero to three.

In a class of the invention, R¹ is hydrogen. In another class of the invention, R² is hydrogen. In another class of the invention, R¹ and R² can be taken together with the carbon atom to which they are attached to form a C₃₋₈ cycloalkyl ring wherein said cycloalkyl ring is optionally substituted with one or two substituents independently selected from C₁₋₆ alkyl, hydroxyalkyl, haloalkyl, aryl, heteroaryl, heterocyclyl or halo.

In a class of the invention, R³ is hydrogen.

In a class of the invention, R⁴ is C₁₋₆ alkyl wherein said alkyl group is optionally substituted with C₃₋₆ cycloalkyl, aryl or halo.

In a class of the invention, R⁵ is hydrogen.

In a class of the invention, R⁶ is C₁₋₆ haloalkyl.

Reference to the preferred embodiments set forth above is meant to include all combinations of particular and preferred groups unless stated otherwise.

Specific embodiments of the papain family cysteine protease inhibitors of the present invention include, but are not limited to:
N¹-(cyanomethyl)-N²-(2,2,2-trifluoro-1-phenylethyl)-L-leucinamide;
N¹(cyanomethyl)-N²{(1S)-2,2,2-trifluoro-1-[4′-(methylsulfonyl)-1,1′-biphenyl-4-yl]ethyl}-L-leucinamide;
N¹-[(1S)-1-cyano-2-phenylethyl]-N²-[(1S)-1-(2′,6′-difluorobiphenyl-4-yl)-2,2-difluoroethyl]-L-leucinamide;
N¹-[(1R,2R)-1-cyano-2-phenylcyclopropyl]-N²-[(1S)-1-(2′,6′-difluorobiphenyl-4-yl)-2,2,2-trifluoroethyl]-L-leucinamide;
N¹-[(1S,2S)-1-cyano-2-phenylcyclopropyl]-N²-[(1S)-1-(2′,6′-difluorobiphenyl-4-yl)-2,2,2-trifluoroethyl]-L-leucinamide;
N¹-[(1S,2S)-1-cyano-2-phenylcyclopropyl]-N²-[(1S)-1-(2′,6′-difluorobiphenyl-4-yl)-2,2,2-trifluoroethyl]-L-leucinamide;
N¹-[(1S)-1-cyano-2-phenylethyl]-N²-{(1S)-2,2,2-trifluoro-1-[4′-(methylsulfonyl)biphenyl-4-yl]ethyl}-L-leucinamide;
N¹-[(1R)-1-cyano-2-(methylsulfonyl)ethyl]-N²-{(1S)-2,2,2-trifluoro-1-[4′-(methylsulfonyl)biphenyl-4-yl]ethyl}-L-leucinamide;
N¹-(1-cyanocyclopropyl)-N²-{(1S)-2,2,2-trifluoro-1-[4-(6-methylpyridin-2-yl)phenyl]ethyl}-L-leucinamide;
N¹-[(1S)-1-cyano-3-(methylthio)propyl]-N²-{(1S)-2,2,2-trifluoro-1-[4′-(methylsulfonyl)biphenyl-4-yl]ethyl}-L-leucinamide;
(2S)-4,4-dichloro-N-(1-cyanocyclopropyl)-2-({(1S)-2,2,2-trifluoro-1-[4′-(methylsulfonyl)biphenyl-4-yl]ethyl}amino)butanamide;
N¹-[(1S)-1-cyano-3-(methylsulfonyl)propyl]-N²-{(1S)-2,2,2-trifluoro-1-[4′-(methylsulfonyl)biphenyl-4-yl]ethyl}-L-leucinamide;
N¹-(1-cyanocyclopropyl)-N²-[(1S)-1-(4′-{1-[(cyclopropylamino)carbonyl]cyclopropyl}biphenyl-4-yl)-2,2,2-trifluoroethyl]-L-leucinamide;
N¹-(1-cyanocyclopropyl)-4-fluoro-N²-{(1S)-2,2,2-trifluoro-1-[4′-methoxy-3′-(methylsulfonyl)biphenyl-4-yl]ethyl}-L-leucinamide;
N¹-[cyano(phenyl)methyl]-N²-{(1S)-2,2,2-trifluoro-1-[4′-(methylsulfonyl)biphenyl-4-yl]ethyl}-L-leucinamide;
   or a pharmaceutically acceptable salt, stereoisomer or N-oxide derivative thereof.

In a class of the invention, the papain family cysteine protease inhibitors of the present invention are administered once weekly, biweekly, twice monthly or once monthly.

Also included within the scope of the present invention is a pharmaceutical composition which is comprised of a compound of Formula I as described above and a pharmaceutically acceptable carrier. The invention is also contemplated to encompass a pharmaceutical composition which is comprised of a pharmaceutically acceptable carrier and any of the compounds specifically disclosed in the present application. These and other aspects of the invention will be apparent from the teachings contained herein.

### Utilities

The use of cysteine protease inhibitors for the treatment of Chagas disease and African trypanosomaisis has been discussed in the art. Substantiation of this hypothesis has been provided by the observation that irreversible inhibitors of cruzipain can cure Chagas disease in mouse models, see Engel, J., et al, J. Exp. Chem., 188, 725-734, 1998. Cruzipain has been reported to exist in at least two polymorphic sequences, known as cruzipain 1 and cruzipain 2, both of which may be involved in the viability of Trypanosoma cruzi (Lima, et al, Molecular & Parasitology 114, 41-52, 2001). A similar role for the cysteine protease trypanopain-Tb has been proposed in the life-cycle of Trypanosoma brucei, the parasite responsible for African trypanosomaisis, or sleeping sickness.

The use of cysteine protease inhibitors for the treatment of malaria has been discussed in the art. Anti-malarial activity has been found with irreversible falcipain inhibitors in a mouse model of malaria (P. vinckei infection), see Olson, J. E., et al, Biorg. Med. Chem., 7, 633-638, 1999.

Two cysteine proteases, SmCL1 and SmCL2, are present in the human blood fluke Schistosoma mansoni. SmCL1 may play a role in the degradation of host hemoglobin, while SmCL2 may be important to the reproductive system of the parasite, see Brady, C. P., et al, Archives of Biochemistry and Biophysics, 380, 46-55, 2000. Thus, inhibition of one or both of these proteases may provide an effective treatment for human schistosomiasis.

LmajcatB and CP2.8ΔCTE are important cysteine proteases of the parasitic protazoa Leishmania major and Leishmania mexicanus respectively, see Alves, L. C., et al, Eur. J. Biochem, 268, 1206-1212, 2001. Thus, inhibition of these enzymes may provide a useful treatment for leishmaniasis.

Exemplifying the invention is the use of any of the compounds described above in the preparation of a medicament for the treatment and prevention of Chagas disease, toxoplasmosis, malaria, African trypanosomiasis, leishmaniasis or schistosomiasis in a mammal in need thereof.

The compounds of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

In the case of tablets for oral use, carriers which are commonly used include lactose and corn starch, and lubricating agents, such as magnesium stearate, are commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The instant compounds are also useful in combination with known agents useful for treating or preventing parasitic diseases, including toxoplasmosis, malaria, African trypanosomiasis, Chagas disease, leishmaniasis or schistosomiasis. Combinations of the presently disclosed compounds with other agents useful in treating or preventing parasitic diseases are within the scope of the invention. A person of ordinary skill in the art would be able to discern which combinations of agents would be useful based on the particular characteristics of the drugs and the disease involved.

Existing therapies for Chagas Disease include, but are not limited to: nifurtimox, benznidazole, allopurinol. Drugs that may have an effect on the parasite include but are not limited to: terbinafine, lovastatin, ketoconazole, itraconazole, posaconazole, miltefosine, ilmofosine, pamidronate, alendronate, and risedronate. Other mechanisms being explored for the treatment of Chagas Disease include, but are not limited to: inhibitors of trypanothione reductase and inhibitors of hypoxanthine-guanine phosphoribosyl transferase (HGPRT), See, Urbina, Current Pharmaceutical Design, 8, 287-295, 2002)

Existing therapies for malaria include, but are not limited to: chloroquine, proguanil, mefloquine, quinine, pyrimethamine-sulphadoxine, doxocycline, berberine, halofantrine, primaquine, atovaquone, pyrimethamine-dapsone, artemisinin and quinhaosu.

Existing therapies for leishmaniasis include, but are not limited to: meglumine antimonite, sodium stibogluconate and amphotericin B.

Existing therapies for schistosomiasis include, but are not limited to: praziquantel and oxamniquine.

Existing therapies for African trypanosomiasis include, but are not limited to: pentamidine, melarsoprol, suramin and eflornithine.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

The terms “once weekly” and “once-weekly dosing,” as used herein, means that a unit dosage, for example a unit dosage of a cruzipain inhibitor, is administered once a week, i.e., once during a seven-day period, preferably on the same day of each week. In the once-weekly dosing regimen, the unit dosage is generally administered about every seven days. A non-limiting example of a once-weekly dosing regimen would entail the administration of a unit dosage of the cruzipaininhibitor every Sunday. It is customarily recommended that a unit dosage for once-weekly administration is not administered on consecutive days, but the once-weekly dosing regimen can include a dosing regimen in which unit dosages are administered on two consecutive days falling within two different weekly periods.

By “biweekly” dosing is meant that a unit dosage of the cruzipain inhibitor is administered once during a two week period, i.e. one time during a fourteen day period, preferably on the same day during each two week period. In the twice-weekly dosing regimen, each unit dosage is generally administered about every fourteen days. A nonlimiting example of a biweekly dosing regimen would entail the administration of a unit dosage of the cruzipain inhibitor every other Sunday. It is preferred that the unit dosage is not administered on consecutive days, but the biweekly dosing regimen can include a dosing regimen in which the unit dosage is administered on two consecutive days within two different biweekly periods.

By “twice monthly” dosing is meant that a unit dosage of the cruzipaininhibitor is administered twice, i.e. two times, during a monthly calendar period. With the twice monthly regimen, the doses are preferably given on the same two dates of each month. In the twice monthly dosing regimen, each unit dosage is generally administered about every fourteen to sixteen days. A nonlimiting example of a twice monthly dosing regimen would entail dosing on or about the first of the month and on or about the fifteenth, i.e. the midway point, of the month. It is preferred that the unit dosages are not administered on the same or consecutive days but the twice-monthly dosing regimen can include a dosing regimen in which the unit dosages are administered on two consecutive days within a monthly period, or different monthly periods. The twice monthly regimen is defined herein as being distinct from, and not encompassing, the biweekly dosing regimen because the two regimens have a different periodicity and result in the administration of different numbers of dosages over long periods of time. For example, over a one year period, a total of about twenty four dosages would be administered according to the twice monthly regimen (because there are twelve calendar months in a year), whereas a total of about twenty six dosages would be administered according to the biweekly dosing regimen (because there are about fifty-two weeks in a year).

The term “once monthly” is used in accordance with the generally accepted meaning as a measure of time amounting to approximately four weeks, approximately 30 days or 1/12 of a calendar year.

The present invention also encompasses a pharmaceutical composition useful in the treatment of parasitic diseases, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for a parasitic disease. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittant throughout the dosage regimen.

In another exemplary application, oral dosages of the present invention, when used for the indicated effects, will range between about 0.01 mg per kg of body weight per week (mg/kg/week) to about 10 mg/kg/week, preferably 0.1 to 10 mg/kg/week, and most preferably 0.1 to 5.0 mg/kg/week. For oral administration, the compositions are preferably provided in the form of tablets containing 2.5 mg, 3.5 mg, 5 mg, 10 mg, 20 mg, 25 mg, 35 mg, 40 mg, 50 mg, 80 mg, 100 mg, 200 mg, 400 mg, and 500 mg of the active ingredient for the symptomatic adjustment of the dosage to the patient to be treated. A medicament typically contains from about 2.5 mg to about 200 mg of the active ingredient, specifically, 2.5 mg, 3.5 mg, 5 mg, 10 mg, 20 mg, 25 mg, 35 mg, 40 mg, 50 mg, 80 mg, 100 mg, 200 mg, 400 mg and 500 mg of active ingredient. Advantageously, the papain family cysteine protease inhibitor may be administered in a single weekly dose. Alternatively, the papain family cysteine protease inhibitor may be administered in a biweekly, twice monthly or monthly dose.

The compounds of the present invention can be used in combination with other agents useful for treating parasitic diseases. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly.

These and other aspects of the invention will be apparent from the teachings contained herein.

### DEFINITIONS

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

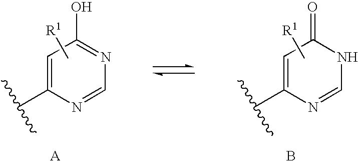

When any variable (e.g. R¹, R², R^{a} etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring carbon atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear, branched, or cyclic arrangement. For example, “C₁-C₁₀ alkyl” specifically includes methyl, ethyl, propyl, butyl, pentyl, hexyl, heptyl, octyl, nonyl, decyl, and so on. “Alkoxy” represents an alkyl group of indicated number of carbon atoms attached through an oxygen bridge.

The term “cycloalkyl” or “carbocycle” shall mean cyclic rings of alkanes of three to eight total carbon atoms, or any number within this range (i.e., cyclopropyl, cyclobutyl, cyclopentyl, cyclohexyl, cycloheptyl or cyclooctyl).

If no number of carbon atoms is specified, the term “alkenyl” refers to a nonaromatic hydrocarbon radical, straight or branched, containing from 2 to 10 carbon atoms and at least 1 carbon to carbon double bond. Preferably 1 carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “cycloalkenyl” shall mean cyclic rings of 3 to 10 carbon atoms and at least 1 carbon to carbon double bond (i.e., cycloprenpyl, cyclobutenyl, cyclopenentyl, cyclohexenyl, cycloheptenyl or cycloocentyl).

The term “alkynyl” refers to a hydrocarbon radical straight or branched, containing from 2 to 10 carbon atoms and at least 1 carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃) CH₂CH(CH₃)Ph, and so on.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or tricyclic ring of up to 10 atoms in each ring, wherein at least one ring is aromatic and contains from 1 to 4 heteroatoms selected from the group consisting of O, N and S. Heteroaryl groups within the scope of this definition include but are not limited to benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo” or “halogen” as used herein is intended to include chloro, fluoro, bromo and iodo. The term “keto” means carbonyl (C═O). The term “alkoxy” as used herein means an alkyl portion, where alkyl is as defined above, connected to the remainder of the molecule via an oxygen atom. Examples of alkoxy include methoxy, ethoxy and the like.

The term “haloalkyl” includes an alkyl portion, where alkyl is as defined above, which is substituted with one to five halo.

The term “arylalkyl” includes an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, and chlorophenylethyl. Examples of alkylaryl include, but are not limited to, toluoyl, ethylphenyl, and propylphenyl.

The term “heteroarylalkyl” as used herein, shall refer to a system that includes a heteroaryl portion, where heteroaryl is as defined above, and contains an alkyl portion. Examples of heteroarylalkyl include, but are not limited to, thienylmethyl, thienylethyl, thienylpropyl, pyridylmethyl, pyridylethyl and imidazoylmethyl.

The term “hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, and the like.

The term “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like.

If the heterocycle contains a nitrogen atom, it is understood that the corresponding N-oxides thereof are also encompassed by this definition.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

The alkyl, alkenyl, alkynyl, cycloalkyl, aryl, heteroaryl and heterocyclyl substituents may be unsubstituted or unsubstituted, unless specifically defined otherwise. For example, a (C₁-C₆)alkyl may be substituted with one or more substituents selected from OH, oxo, halogen, alkoxy, dialkylamino, or heterocyclyl, such as morpholinyl, piperidinyl, and so on. In the case of a disubstituted alkyl, for instance, wherein the substituents are oxo and OH, the following are included in the definition: —(C═O)CH₂CH(OH)CH₃, —(C═O)OH, —CH₂(OH)CH₂CH(O), and so on.

Whenever the term “alkyl” or “aryl” or either of their prefix roots appear in a name of a substituent (e.g., aryl C₀₋₈ alkyl) it shall be interpreted as including those limitations given above for “alkyl” and “aryl.” Designated numbers of carbon atoms (e.g., C₁₋₁₀) shall refer independently to the number of carbon atoms in an alkyl or cyclic alkyl moiety or to the alkyl portion of a larger substituent in which alkyl appears as its prefix root.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci., 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

For purposes of this specification, the following abbreviations have the indicated meanings:
AcOH=acetic acid
Boc=t-butyloxycarbonyl
Boc₂O=di-tert-butyl dicarbonate
BuLi=butyl lithium
CCl₄=carbon tetrachloride

CH₂Cl₂=methylene chloride
CH₃CN=acetonitrile
CHCl₃=chloroform
Cs₂CO₃=cesium carbonate
CuI=copper iodide
DMA=N,N-dimethyl acetamide
DMAP=4-(dimethylamino)pyridine
DMF=N,N-dimethylformamide
DMSO=dimethylsulfoxide
EDCI=1-(3-dimethylaminopropyl)-3-ethylcarbodiimide hydrochloride
Et₂O=diethyl ether
Et₃N=triethylamine
EtOAc=ethyl acetate
EtOH=ethanol
HATU=o-(7-azabenzotriazol-1-yl)-N,N,N′,N′-tetramethyluronium hexafluorophosphate
HOAc=acetic acid
K₂CO₃=potassium carbonate
KOBu^{t}=potassium tert-butoxide
LiOH=lithium hydroxide
mCPBA=metachloroperbenzoic acid
MeOH=methanol
MeSO₃H=methane sulfonic acid
MgSO₄=magnesium sulfate
Ms=methanesulfonyl=mesyl
MsCl=methanesulfonyl chloride
NaBH₄=sodium borohydride
NaH=sodium hydride
Na₂CO₃=sodium carbonate
NaHCO₃=sodium hydrogencarbonate
NaOH=sodium hydroxide
Na₂SO₄=sodium sulfate
NBS=N-bromosuccinimide
NH₃=ammonia
NH₄Cl=ammonium chloride
Pd/C=palladium on carbon
PdCl₂(dppf)=[1,1′-bis(diphenylphosphino)ferrocene]dichloropalladium(II)
Pd₂(dba)₃=tris(dibenzylideneacetone)dipalladium(0)
PG=protecting group
PPh₃=triphenylphosphine
PPTS=pyridinium p-toluenesulfonate
iPr₂Nli=lithium diisopropyl amide
PyBOP=benzotriazol-1-yloxytris(pyrrolidino)phosphonium-hexafluorophosphate
rt=room temperature
sat. aq.=saturated aqueous
TFA=trifluoroacetic acid
THF=tetrahydrofuran
tlc=thin layer chromatography
Me=methyl
Et=ethyl
n-Pr=normal propyl
i-Pr=isopropyl
n-Bu=normal butyl
i-Bu=isobutyl
s-Bu=secondary butyl
t-Bu=tertiary butyl

The compounds of the present invention can be prepared according to the following general procedures using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

### Schemes

Compounds of the present invention can be prepared according to Scheme 1, as indicated below. Thus an α-amino ester may be added to a haloalkyl ketone to form an aminal which may be dehydrated to an imine in the presence of a dehydrating agent such as TiCl₄, MgSO₄ or isopropyl trifluoroacetate. Reduction of the imine with a reducing agent such as sodium cyanoborohydride or sodium borohydride provides the amine. Ester hydrolysis and amide formation with an appropriately substituted aminoacetonitrile provides compounds of the current invention. If the substituent on D system is a halogen, a palladium-catalyzed Suzuki coupling with an appropriate boronic acid provides additional compounds of the current invention.

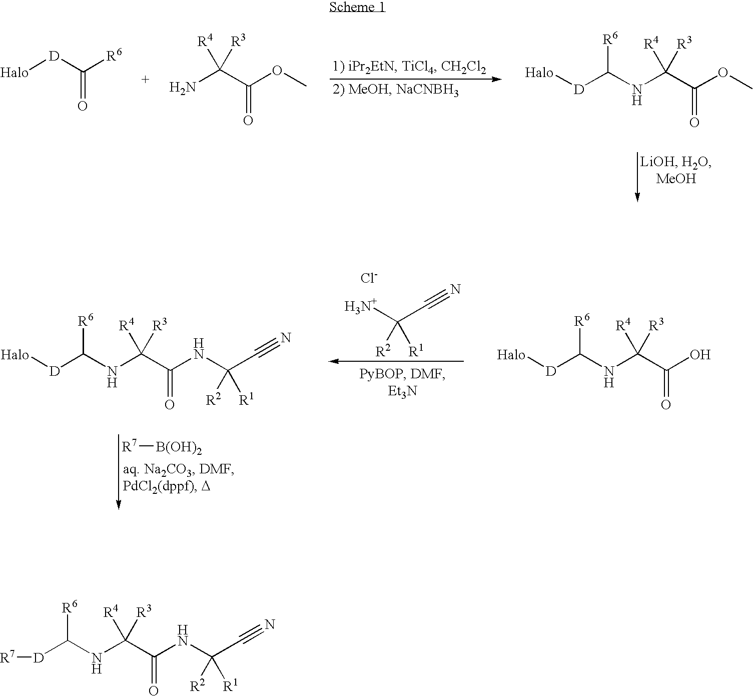

Compounds of the present invention may also be prepared according to Scheme 2, as indicated below. A haloalkylketone or aldehyde may be condensed with an amino alcohol to give a cyclic aminal. Treatment with 3 equivalents of a Grignard reagent or organolithium reagent will provide the appropriate alkylated amino alcohol. Oxidation of the alcohol with a chromium system such as a Jones oxidation, or alternatively by a two-step oxidation (eg oxalyl chloride/DMSO/Et₃N followed by NaClO) will provide the corresponding carboxylic acid. Peptide coupling and Suzuki reaction as described in Scheme 1 will provide compounds of the current invention.

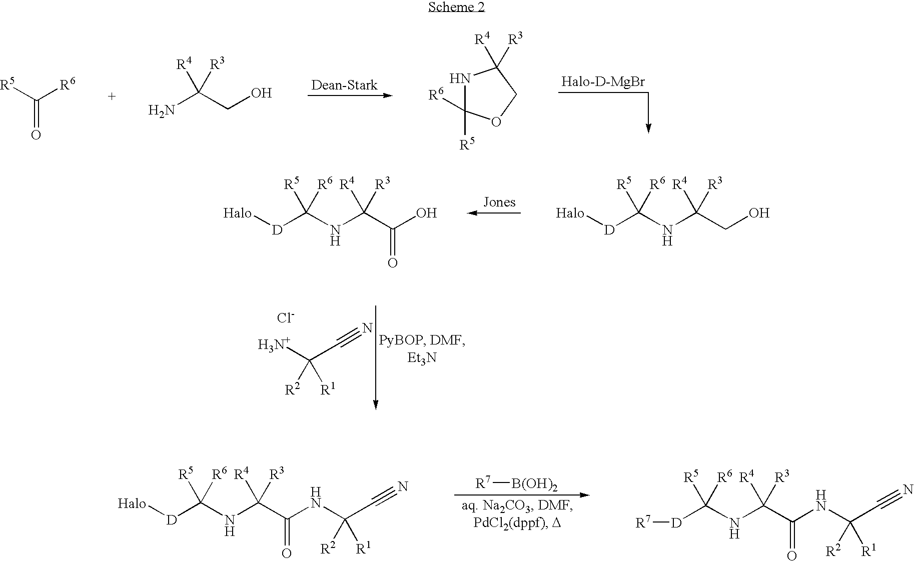

Compounds of the present invention may also be prepared according to Scheme 3, as indicated below. A haloalkylketone or aldehyde may be condensed with an amino alcohol to give an acyclic aminal. Treatment with multiple equivalents of a Grignard reagent or organolithium reagent will provide the appropriate alkylated amino alcohol. This alcohol can be converted into compounds of the current invention by the method described in Scheme 2.

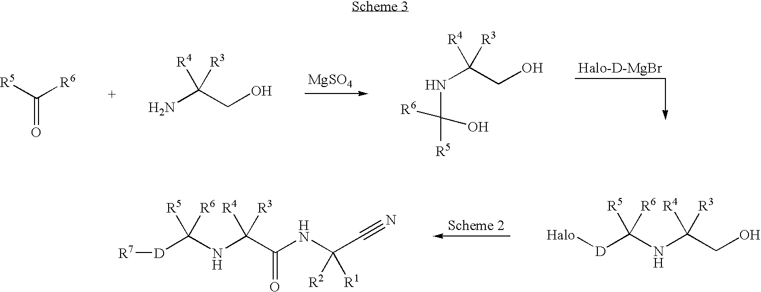

Compounds of the current invention may also be prepared according to Scheme 4, as indicated below. A hemiacetal may be condensed with an amino alcohol in which the alcohol moiety is protected with a suitable protecting group. Treatment of the resulting imine with a Grignard reagent or organolithium reagent will provide the appropriate alkylated amino alcohol. The alcohol protecting group can then be removed and the alcohol can be converted into compounds of the current invention either by the method described in Scheme 2 or by first conducting the Suzuki reaction, followed by oxidizing the alcohol with H₅IO₆/CrO₃ and then peptide coupling.

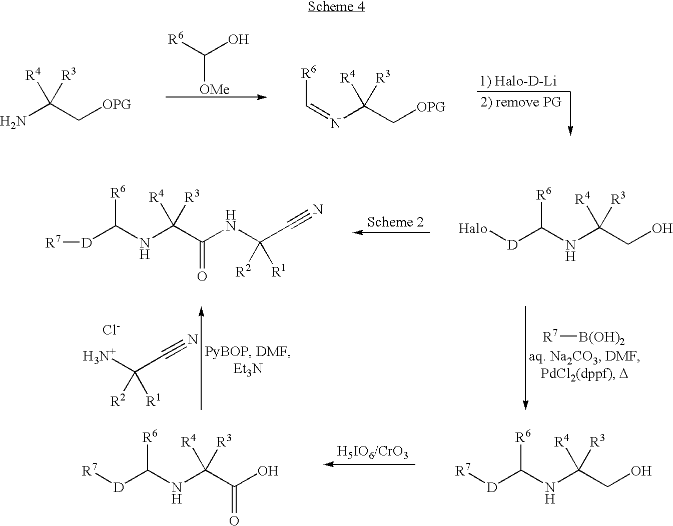

The following examples describe the synthesis of selected compounds of the present invention.

### Example 1

### Synthesis of N¹-(cyanomethyl)-N²-(2,2,2-trifluoro-1-phenylethyl)-L-leucinamide

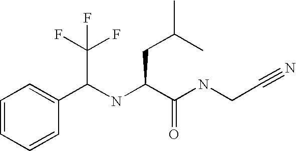

To a solution of L-leucine methyl ester hydrochloride (975 mg, 5.37 mmol) in dichloromethane (30 mL) was added 2,2,2-trifluoroacetophenone (0.75 mL, 5.34 mmol) and diisopropylethylamine (3.5 mL, 20 mmol). TiCl₄ (0.55 mL, 5.0 mmol) in 0.45 mL dichloromethane was added dropwise, and the mixture was stirred overnight. Additional TiC₄ (0.4 mL, 3.6 mmol) was then added and the mixture was stirred 3 h. A solution of NaCNBH₃ (1050 mg, 16.7 mmol) in MeOH (20 mL) was added and the mixture was stirred 2 h. Poured into 1N NaOH and extracted with ethyl acetate (2×). The organic phase was washed with 1N NaOH and brine, then dried over MgSO₄ and evaporated. Purification by ISCO column chromatography (gradient 30% to 90% ethyl acetate/hexanes) provided methyl N-(2,2,2-trifluoro-1-phenylethyl)-L-leucinate.

To a room temperature solution of methyl N-(2,2,2-trifluoro-1-phenylethyl)-L-leucinate (150 mg, 0.50 mmol) in 2:1 THF/MeOH was added 1M LiOH. The mixture was stirred overnight and concentrated. The residue was partitioned between ethyl acetate and pH 3.5 phosphate buffer. The organic phase was washed with brine, dried over MgSO₄ and concentrated to give N-(2,2,2-trifluoro-1-phenylethyl)-L-leucine.

A mixture of N-(2,2,2-trifluoro-1-phenylethyl)-L-leucine (149 mg, 0.50 mmol), aminoacetonitrile hydrochloride (102 mg, 1.1 mmol) and PyBOP (260 mg, 0.50 mmol) was dissolved in DMF (5 mL). Triethylamine (0.3 mL, 2.1 mmol) was added and the mixture was stirred overnight, then poured into pH 3 phosphate buffer and extracted with 3:1 ether/ethyl acetate. The organic phase was washed with saturated aqueous NaHCO₃ and brine, dried over MgSO₄ and evaporated. Purification by ISCO column chromatography (gradient 20% to 50% ethyl acetate/hexanes) provided N¹-(cyanomethyl)-N²-(2,2,2-trifluoro-1-phenylethyl)-L-leucinamide as a 1:1 mixture of diastereomers. MS (+APCI): 313.9 [M+1].

### Example 2

### Synthesis of N¹(cyanomethyl)-N²{(1S)-2,2,2-trifluoro-1-[4′-(methylsulfonyl)-1,1′-biphenyl-4-yl]ethyl}-L-leucinamide

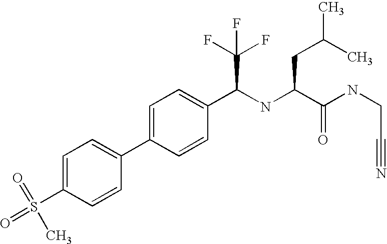

### Step 1: Preparation of (2S)-1-{[tert-butyl(dimethyl)silyl]oxy}-4-methylpentan-2-amine

To a room temperature dichloromethane (100 mL) solution of L-leucinol (6.0 g) was added triethylamine (11 mL), DMAP (0.1 g) and t-butyldimethylsilyl chloride (8.5 g). The mixture was stirred at room temperature for 2 hours and then water was added. The organic layer was separated and the aqueous further extracted with dichloromethane. The combined organic layers were washed with brine, dried with magnesium sulfate and the solvent was removed in vacuo to yield the title compound, a residue which was used as such in the next reaction.

¹H NMR (CD₃COCD₃) δ 3.48 (m, 2H), 3.32 (m, 1H), 2.76 (m, 1H), 1.78 (m, 1H), 1.22-1.02 (m, 2H), 0.88 (m, 15H), 0.06 (s, 6H).

### Step 2: Preparation of (2S)-1-{[tert-butyl(dimethyl)silyl]oxy}-4-methyl-N-[(1E)-2,2,2-trifluoroethylidenelpentan-2-amine

A toluene (300 mL) solution of (2S)-1-{[tert-butyl(dimethyl)silyl]oxy}-4-methylpentan-2-amine from Step 1 (50 g) and trifluoroacetaldehyde methyl hemiacetal (35 mL) was heated to reflux for 16 hours during which time water was collected in a Dean-Stark trap. The solvent was evaporated in vacuum and the residue was purified on SiO₂ using hexanes and ethyl acetate (9:1) as eluant to yield the title compound.

¹H NMR (CD₃COCD₃) δ 7.88 (m, 1H), 3.76-3.45 (m, 3H), 1.60-1.25 (m, 3H), 0.88 (m, 15H), 0.06 (s, 3H), 0.04 (s, 3H).

### Step 3: Preparation of (2S)-2-{[(1S)-1-(4-bromophenyl)-2,2,2-trifluoroethyl]amino}-4-methylpentan-1-ol

n-BuLi (2.5 M in hexanes, 42 mL) was added to a −70° C. THF (400 mL) solution of 1,4-dibromobenzene (25.8 g) and the mixture was stirred for 25 minutes. A THF (30 mL) solution of (2S)-1-{[tert-butyl(dimethyl)silyl]oxy}-4-methyl-N-[(1E)-2,2,2-trifluoroethylidene]pentan-2-amine (31 g) was then added dropwise and the mixture was stirred for 1.5 hour. It was then poured slowly into a mixture of ethyl acetate (500 mL), water (2 L), ice (300 g) and ammonium chloride (100 g) under vigorous stirring. The organic layer was separated and the aqueous further extracted with ethyl acetate (2×500 mL). The combined organic layers were washed with brine, dried with magnesium sulfate and the solvent was removed in vacuo to yield a residue, which was used as such. The residue from above was dissolved in THF (250 mL) and the solution was cooled to 0° C. A 1 M THF solution of t-butylammonium fluoride (110 mL) was added dropwise and the mixture was reacted for 4 hours. It was poured into ethyl acetate (300 mL), water (2 L) and ammonium chloride (100 g) under vigorous stirring. The organic layer was separated and the aqueous further extracted with ethyl acetate (2×100 mL). The combined organic layers were washed with brine, dried with magnesium sulfate and the solvent was removed in vacuo to yield a residue which was purified on SiO₂ using a gradient of ethyl acetate and hexanes (1:5 to 1:4) as eluant to yield the title compound.

¹H NMR (CD₃COCD₃) δ 7.6 (2H, d), 7.45 (2H, d), 4.55 (1H, m), 3.65-3.7 (1H, m), 3.5-3.55 (1H, m), 3.25-3.35 (1H, m), 2.6-2.7 (1H, m), 2.25-2.35 (1H, m), 1.65-1.75 (1H, m), 1.3-1.4 (1H, m), 1.2-1.3 (1H, m), 0.75-0.9 (6H, dd).

### Step 4: Preparation of (2S)-4-methyl-2-({(1S)-2,2,2-trifluoro-1-[4′-(methylthio)-1,1′-biphenyl-4-yl]ethyl}amino)pentan-1-ol

A stream of nitrogen was passed through a suspension made of the bromide from Step 3 (27.7 g), 4-(methylthio)phenylboronic acid (15.7 g), 2 M Na₂CO₃ (100 mL) and n-propanol (500 mL) for 15 minutes. A 1:3 mixture (3.5 g) of Pd(OAc)₂ and PPh₃ was then added and the reaction was warmed to 70° C. and stirred under nitrogen for 8 hours. The mixture was cooled to room temperature, diluted with ethylacetate (500 mL) and poured over water (2 L) and ice (500 g). The ethyl acetate layer was separated and the aqueous further extracted with ethyl acetate (200 mL). The combined ethyl acetate extracts were washed with 0.5 N NaOH (2×200 mL), with aqueous NH₄Cl, brine and dried with magnesium sulfate. Removal of the solvent left a residue that was purified by chromatography on SiQ₂ using a gradient of ethyl acetate and hexanes (1:4 to 1:3) and again with acetone and toluene (1:10). The residue was dissolve in hot hexanes (200 mL) and the solution was allowed to cool to 0° C. under stirring. The obtained solid was filtered and dried to yield the title compound.

¹H NMR (CD₃COCD₃) δ 7.7 (2H, d), 7.65 (2H, d), 7.6 (2H, d), 7.35 (2H, d), 4.5-4.6 (1H, m), 3.7 (1H(OH), m), 3.5-3.6 (1H, m), 3.3-3.4 (1H, m), 2.7 (1H, m), 2.5 (3H, s), 2.3-2.4 (1H(NH), m), 1.65-1.75 (1H, m), 1.2-1.4 (3H, m), 0.8-0.9 (6H, dd).

### Step 5: Preparation of (2S)-4-methyl-2-({(1S)-2,2,2-trifluoro-1-[4′-(methylsulfonyl)-1,1′-biphenyl-4-yl]ethyl}amino)pentan-1-ol

To a 0° C. solution of the sulfide (19 g) from Step 4 in toluene (400 mL) was added Na₂WO₄.2H₂O (0.16 g) and Bu₄NHSO₄ (0.81 g). Then 30% hydrogen peroxide (12.2 mL) was slowly added and the mixture was stirred at room temperature for 4.5 hours. The mixture was poured slowly on a mixture of ice, dilute aqueous sodium thiosulfate and ethyl acetate. The organic layer was separated and the aqueous further extracted with ethyl acetate (2×100 mL). The combined organic layers were washed with brine, dried with magnesium sulfate and the solvent were removed in vacuo to yield a residue which was purified on SiO₂ using ethyl acetate and hexanes (1:1) as eluant to yield the product.

¹H NMR (CD₃COCD₃) δ 8.05 (2H, d), 8.0 (2H, d), 7.85 (2H, d), 7.7 (2H, d), 4.6-4.7 (1H, m), 3.75 (1H, m), 3.6 (1H, m), 3.35-3.45 (1H, m), 3.2 (3H, s), 2.7-2.8 (1H, m), 2.35-2.45 (1H, m), 1.7-1.8 (1H, m), 1.2-1.5 (2H, m), 0.8-0.95 (6H, dd).

### Step 6: Preparation of N-{(1S)-2,2,2-trifluoro-1-[4′-(methylsulfonyl)-1,1′-biphenyl-4-yl]ethyl}-L-leucine

A suspension of H₅IO₆/CrO₃ (529 mL of 0.44 M in CH3CN; see Note below) was cooled to 0° C. and a solution of the alcohol from Step 5 (20 g) in CH₃CN (230 mL) was added dropwise. The mixture was stirred at 0-5° C. for 3.5 hours. It was poured into pH 4 Na₂HPO₄ (1.5 L) under vigorous stirring and the mixture was extracted with diethyl ether (3×250 mL). The combined ether extracts were washed with water and brine (1:1), with dilute aqueous NaHSO₃ and brine. The organic extract was dried with sodium sulfate, filtered and the solvents were evaporated to dryness to yield a residue that was split into two batches for the following purification.

The crude acid from above (10 g) was dissolved in isopropyl acetate (250 mL) and extracted into cold 0.1 N NaOH (3×250 mL). The combined extracts were washed with diethyl ether (250 mL) and then slowly acidified with 6 N HCl to pH 4. The carboxylic acid was extracted with isopropyl acetate (2×250 mL) and the isopropyl acetate layer dried and concentrated to yield the product essentially pure and used as such in the next step.

Note: The oxidizing reagent (H₅IO₆/CrO₃) was prepared as described in Tetrahedron Letters 39 (1998) 5323-5326 but using HPLC grade CH₃CN (contains 0.5% water); no water was added.

¹H NMR (CD₃COCD₃) δ 8.05 (2H, d), 7.95 (2H, d), 7.8 (2H, d), 7.65 (2H, d), 4.45-4.55 (1H, m), 3.55-3.6 (1H, m), 3.2 (3H, s), 2.8-3.0 (broad m, NH/OH) 1.95-2.05 (1H, m), 1.55-1.6 (2H, m), 0.9-1.0 (6H, m).

### Step 7: Preparation of N¹(cyanomethyl)-N²{(1S)-2,2,2-trifluoro-1-[4′-(methylsulfonyl)-1,1′-biphenyl-4-yl]ethyl}-L-leucinamide

To a DMF (200 mL) solution of the acid from Step 7 (9 g) was added benzotriazol-1-yloxytris(dimethylamino)phosphonium hexafluorophosphate (11.6 g), aminoacetonitrile hydrochloride (3.94 g) and the mixture was cooled to 0° C. Triethylamine (9.9 mL) was added dropwise and the mixture warmed to room temperature and stirred for 16 hours. It was poured into ice and saturated aqueous sodium bicarbonate and extracted with diethyl ether (3×100 mL). The combined extracts were washed with brine, dried with magnesium sulfate and the solvent removed in vacuo. The residue was purified by chromatography on SiO₂ using ethyl acetate and hexanes (1:1). The title compound was then stirred in diethyl ether for 16 hours, filtered and dried (mp 140.5° C.).

¹H NMR (CD₃COCD₃) δ 8.0 (2H, d), 7.95 (2H, d), 7.8 (2H, d), 7.65 (2H, d), 4.35-4.45 (1H, m), 4.1-4.2 (2H, m), 3.45-3.55 (1H, m), 3.15 (3H, s), 2.65-2.7 (1H, m), 1.85-1.95 (1H, m), 1.4-1.6 (2H, m), 0.85-0.95 (6H, m).

### Example 3

### Synthesis of N¹-[(1S)-1-cyano-2-phenylethyl]-N²-[(1S)-1-(2′,6′-difluorobiphenyl-4-yl)-2,2-difluoroethyl]-L-leucinamide

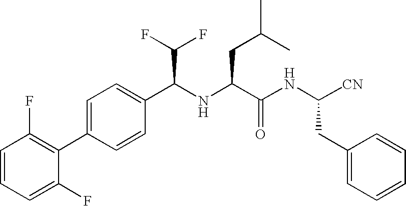

### Step 1: Preparation of methyl N-[(1S)-1-(2′,6′-difluorobiphenyl-4-yl)-2,2-difluoroethyl]-L-leucinate

Methyl N-[(1S)-1-(4-bromophenyl)-2,2-difluoroethyl]-L-leucinate was prepared according to methods described in International Publication No. WO 03/075836, which published on Sep. 18, 2003.

To a solution of methyl N-[(1S)1-(4-bromophenyl)2,2-difluoroethyl]-L-leucinate (164 mg, 0.45 mmol) in DMF (4.5 mL) was added bis(pinacolato)diboron (137 mg, 0.54 mmol), PdCl₂dppf (11 mg, 0.013 mmol) and KOAc (132 mg, 1.35 mmol). The mixture was placed under a nitrogen atmosphere, then heated with microwave irradiation to 120° C. for 10 min. The mixture was cooled and partitioned between EtOAc and aq. NaHCO₃. The aqueous layer was extracted 3× with EtOAc and the combined organic layers were washed with brine, dried over MgSO₄ and concentrated to provide the crude product. To a solution of this material in DMF (4 mL) was added 1-bromo-2,6-difluorobenzene (260 mg, 1.35 mmol), PdCl₂dppf (11 mg, 0.013 mmol) and aq. Na₂CO₃ (2M solution, 0.68 mL). The mixture was heated with microwave irradiation to 120° C. for 8 min. The mixture was cooled and partitioned between EtOAc and aq. NaHCO₃. The aqueous layer was extracted 3× with EtOAc and the combined organic layers were washed with brine, dried over MgSO₄ and concentrated. Purification by silica gel chromatography (gradient: hexanes to 15% EtOAc:hexanes) provided 150 mg of the title compound.

### Step 2: Preparation of N-[(1S)-1-(2′,6′-difluorobiphenyl-4-yl)-2,2-difluoroethyl]-L-leucine

To a solution of methyl N-[(1S)-1-(2′,6′-difluorobiphenyl-4-yl)-2,2-difluoroethyl]-L-leucinate in a mixture of THF (10 mL), MeOH (2 mL) and water (4 mL) was added lithium hydroxide hydrate (24 mg, 0.565 mmol). The mixture was stirred overnight, then diluted with pH 3 phosphate buffer and concentrated by one third. The residue was extracted twice with dichloromethane which was dried over MgSO₄ and concentrated to provide the title compound.

### Step 3: Preparation of tert-butyl [(1S)-1-cyano-2-phenylethyl]carbamate

To a solution of N-(tert-butoxycarbonyl)-L-phenylalaninamide (1.86 g, 7.0 mmol) in dioxane (30 mL) was added pyridine (3 mL, 35 mmol) and trifluoroacetic anhydride (2 mL, 14 mmol). The mixture was stirred at room temperature overnight, then diluted with 1:1 EtOAc/ether and washed sequentially with aq NaHCO₃, water, 1 M HCl (2×) and brine. The organic phase was dried over MgSO₄ and concentrated to provide the title compound.

### Step 4: Preparation of (2S)-2-amino-3-phenylpropanenitrile

To a solution of tert-butyl [(1S)-1-cyano-2-phenylethyl]carbamate (1.8 g, 7.0 mmol) in dichloromethane (10 mL) was added thioanisole (1.65 mL, 14 mmol) followed by a solution of methanesulfonic acid (0.68 mL, 10.5 mmol) in dichloromethane (3 mL). The mixture was stirred for 1.5 h, giving a suspension. Diethyl ether (50 mL) was added and the title compound was collected by filtration as its mesylate salt.

### Step 5: Preparation of N¹-[(1S)-cyano-2-phenylethyl]-N²-[(1S)-1-(2′,6′-difluorobiphenyl-4-yl)-2,2-difluoroethyl]-L-leucinamide

To a solution of N-[(1S)-1-(2′,6′-difluorobiphenyl-4-yl)-2,2-difluoroethyl]-L-leucine (339 mg, 0.88 mmol) and (2S)-2-amino-3-phenylpropanenitrile methanesulfonate (214 mg, 0.88 mmol) in DMF (5 mL) was added HATU (400 mg, 1.0 mmol) followed by triethylamine (0.40 mL, 2.9 mmol). The mixture was stirred at room temperature overnight, then diluted with aq NaHCO₃ and extracted with 1:1 EtOAc/ether. The organic phase was washed with aq NaH₂PO₄ (3×) and brine, then dried over MgSO₄ and concentrated. Purification by flash chromatography (30% EtOAc:hexanes) provided the title compound.

¹H NMR (d₆-acetone, 500 MHz) δ 8.12 (d, 1H), 7.45 (m, 5H), 7.34 (m, 4H), 7.28 (m, 1H), 7.10 (m, 2H), 6.05 (dt, 1H), 5.08 (m, 1H), 3.82 (m, 1H), 3.40 (m, 1H), 3.17 (m, 1H), 3.08 (m, 1H), 2.32 (m, 1H), 1.80 (m, 1H), 1.42 (m, 2H), 0.88 (m, 6H).

### Example 4

Using the methods described above, the following compounds were prepared:

| Compound | Characterization Data |
|---|---|
| 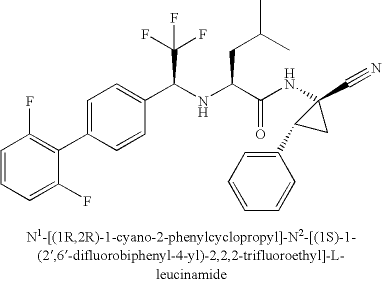 | MS (−ESI): 540 [M − 1] |
| 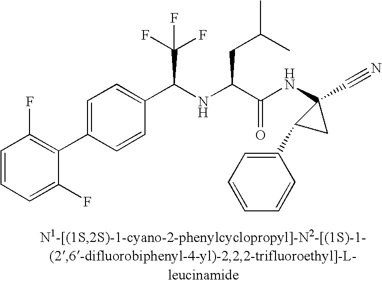 | MS (−ESI): 540 [M − 1] |
| 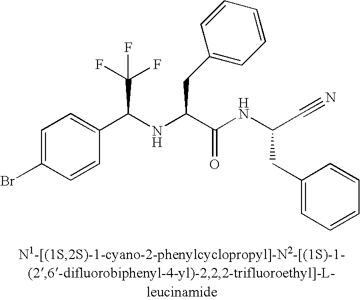 | MS (−APCI): 530.1 [M − 1] |
| 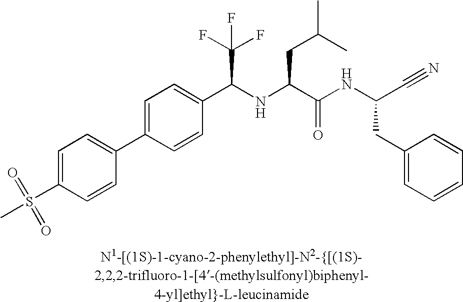 | MS (+ESI): 572 [M + 1]⁺ |
| 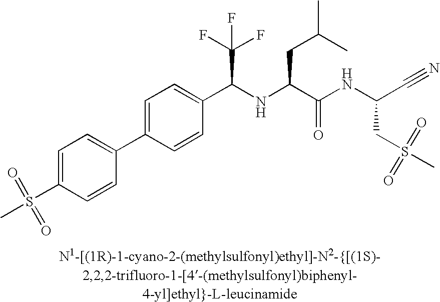 | MS (+ESI): 574 [M + 1]⁺ |
| 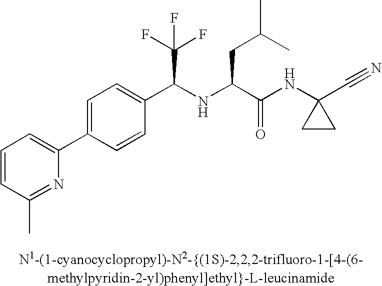 | MS (+ESI): 445.2 [M + 1]⁺; m.p. 188° C. |
| 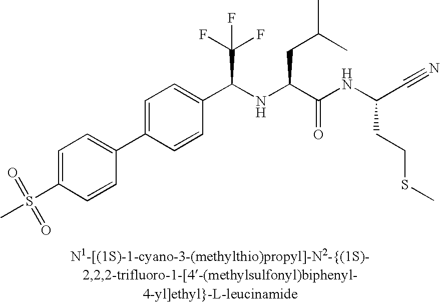 | MS (+ESI): 556 [M + 1]⁺ |
| 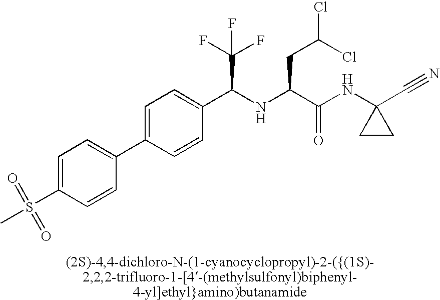 | MS (+ESI): 548, 550 [M + 1]⁺; |
| 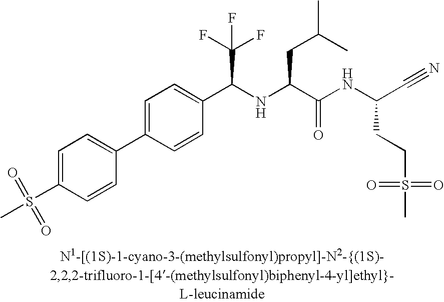 | MS (+ESI): 588 [M + 1]⁺; m.p. 147-148° C. |
| 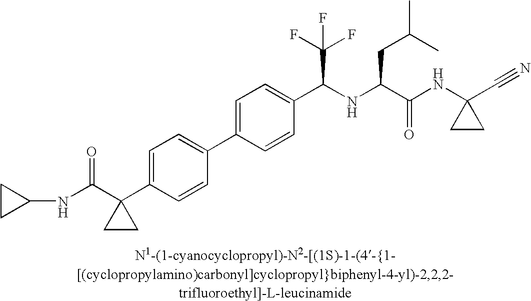 | MS (+APCI): 553.4 [M + 1]⁺ |
| 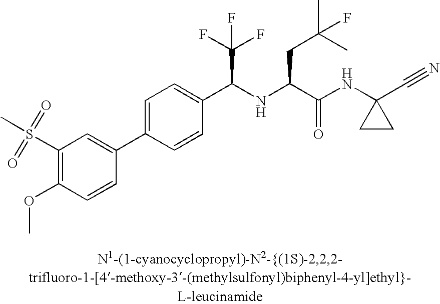 | MS (+ESI): 556.3 [M + 1]⁺ |
| 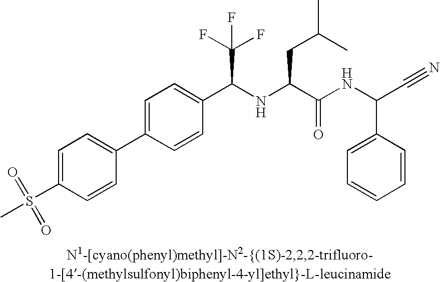 | MS (+ESI): 556 [M + 1] |

### Pharmaceutical Composition

As a specific embodiment of this invention, 100 mg of N¹-(cyanomethyl)N²-[2,2,2-trifluoro-1-(4′-piperazin-1-yl-1,1′-biphenyl-4-yl)ethyl]-L-leucinamide, is formulated with sufficient finely divided lactose to provide a total amount of 580 to 590 mg to fill a size 0, hard-gelatin capsule.

The compounds disclosed in the present application exhibited activity in the following assays. In addition, the compounds disclosed in the present application have an enhanced pharmacological profile relative to previously disclosed compounds.

### Cruzipain Assay

Serial dilutions (1/3) from 500 μM down to 0.0085 μM of test compounds were prepared in dimethyl sulfoxide (DMSO). Then 2 μL of DMSO from each dilution were added to 100 μL of cruzipain (500 ng/mL) in assay buffer solution (NaOAc, 50 mM (pH 5.5); DTT, 5 mM; and DMSO 10% v/v). The assay solutions were mixed for 5-10 seconds on a shaker plate and incubated for 15 minutes at room temperature. Z-Phe-Arg-AMC (20 μM) in 10 μL of assay buffer was added to the assay solutions. Hydrolysis of the coumarin leaving group (AMC) was followed by spectrofluorometry (Exλ=350 nm; Emλ=460 nm) for 10 minutes. Percent of inhibition were calculated by fitting experimental values to standard mathematical model for dose response curve.

### T. cruzi Epimastigotes Assay

The epimastigote form of T. cruzi (Brazilian strain) was initiated in a 25 cm² flask with a cell density of 2×10⁶ epimastigotes per mL and grown in liver infusion tryptose (LIT) broth medium, supplemented with 10% newborn calf serum (Gibco) and antibiotics, at 28° C. with agitation (80 rpm) to a cell density of 0.5×10⁷ to 1×10⁷, measured with an electronic particle counter (model ZBI; Coulter Electronics Inc., Hialeah, Fla.) and by direct counting with a hemocytometer. Test compounds in DMSO were added to the flasks when the epimastigotes cell density reached 0.5×10⁷ to 1×10⁷ per ml then incubated for 24 to 48 h and the epimastigotes harvested during the logarithmic growth phase. The harvested epimastigotes were washed three times with 1M phosphate-buffered saline (PBS; pH 7.4) by centrifugation at 850 g for 15 minutes at 4° C. The harvested epimastigotes were reincubated in fresh LIT broth supplemented with 10% newborn calf serum and antibiotics, at 28° C. with agitation (80 rpm) and the viability of the epimistagotes evaluated for up to one week using trypan blue exclusion (light microscopy) and [³H]-thymidine incorporation assay (see below).

### T. cruzi Trypomastigote Assay

The epimastigote forms of T. cruzi were grown as described above and harvested on day 14 (stationary phase) washed three times in Grace's insect medium pH 6.5 (Invitrogen or Wisent) and induced to the trypomastigote form by metacyclogenesis by the addition of fresh Grace medium supplemented with 10% fetal calf serum (FCS) and haemin (25 μg/ml) and cultured for up to five days at 28° C. To produce more trypomastigotes the culture may be used to infect a monolayer of mammalian cells such as U937 (human macrophage), J774 (mouse macrophage) or Vero (African green monkey kidney) cells up to 4 days. Trypomastigotes released to the supernatant were collected by a 3000 g centrifugation for 15 minutes and washed twice in Hank's balanced salt saline supplemented with 1 mM glucose (HBSS). Test compounds in DMSO were added to the culture of trypomastigotes with a cell density of 10⁶ per mL then incubated in RPMI-10% at 37° C. for 24 to 48 h. The trypomastigotes were harvested and reduction in number (parasite lysis) was determined using a Neubauer chamber and the LD₅₀ value (drug concentration that resulted in a 50% reduction in trypomastigotes when compared to an untreated control) was estimated by plotting percentage of reduction against the logarithm of drug concentration. The viability of the harvested trypomastigotes was evaluated by their ability to infect macrophages and grow in fresh media as determined by a ³H-thymidine incorporation assay (see below).

### T. cruzi Amastigote Activity (Intracellular) Assay

The epimastigotes form of T. cruzi was cultured in Grace's insect medium supplemented with 10% FCS and haemin (25 μg/ml) for up to fourteen days at 28° C. to induce the formation of the metacyclic form, so that about 30% of the parasite cells were in the metacyclic form. These parasite cells were harvested and used to infect confluent mammalian cells such as U937 (human macrophage), J774 (mouse macrophage) or Vero (African green monkey kidney) cell cultures grown in 24 wells microplates in MEM at 37° C. and 5% CO₂. After the parasitic cells were allowed to infect the macrophages, the culture media was removed and the test compounds in MEM culture medium were added to the wells and the microplates incubated for 48 h. At the end of the incubation period the media was removed and the macrogphages were fixed and stained with May Gruinwald Giemsa stain. The number of amastigotes/100 macrophages (No. A/100 Mφ) was determined and the anti-amastigote activity expressed as (% AA):

% AA=[1−(No.A/100Mφ)p/(No.A/100Mφ)c]×100

### ³H-thymidine Incorporation Assay

A 200 μL MEM suspension containing a mammalian cell line such as U937 (human macrophage), J774 (mouse macrophage) or Vero (African green monkey kidney) cells was added to each well in 96 well flat-bottom microtitre plates and incubated for 24 to 48 h at 37° C. in 5% CO₂. The medium was removed and the cells washed three times in PBS. A 200 μL mixture of MEM containing 1×17/mL stationary phase T. cruzi trypomastigotes was added to each well then incubated for 24 or 48 h under the same conditions. After the incubation period the media was removed and the cells washed three times in PBS. The test compounds in MEM were added to the appropriate wells and incubated for up to three days. At the end of the incubation period the media was removed and the cells washed three times in PBS and the macrophages were lysed with 0.01% sodium dodecyl sulphate(SDS) and the parasitic cells harvested. The harvested parasitic cells were suspended in Grace's insect media and incubated at 28° C. for 48 h. At the end of the incubation period 1 μCi of ³H-thymidine in Grace's insect media was added to each well and incubated for an additional 20 h; this was harvested and ³H-thymidine incorporation was measured.

What is claimed is:

1. A method of treating a parasitic disease with a papain family cysteine protease inhibitor.

2. The method of claim 1 wherein the parasitic disease is toxoplasmosis, malaria, African trypanosomiasis, Chagas disease, leishmaniasis or schistosomiasis.

3. The method of claim 1 wherein the papain family cysteine protease inhibitor is a compound of formula I: 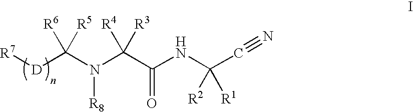
wherein R¹ is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
R² is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, C₃₋₆ cycloalkyl, halo, hydroxyalkyl, hydroxy, alkoxy or keto;
or R¹ and R² can be taken together with the carbon atom to which they are attached to form a C₃₋₈ cycloalkyl ring or heterocyclyl ring wherein said cycloalkyl and heterocycl rings are optionally substituted with one or two substituents independently selected from C₁₋₆ alkyl, hydroxyalkyl, haloalkyl, aryl, heteroaryl, heterocyclyl or halo;
R³ is hydrogen, C₁₋₆ alkyl or C₂₋₆ alkenyl wherein said alkyl and alkenyl groups are optionally substituted with C₃₋₆ cycloalkyl, aryl or halo;
R⁴ is hydrogen, C₁₋₆ alkyl or C₂₋₆ alkenyl wherein said alkyl and alkenyl groups are optionally substituted with C₃₋₆ cycloalkyl, aryl or halo;
or R³ and R⁴ can be taken together with the carbon atom to which they are attached to form a C₃₋₈ cycloalkyl ring, C₅₋₈ cycloalkenyl ring, or five to seven membered heterocyclyl ring wherein said cycloalkyl, cycloalkenyl and heterocyclyl rings are optionally substituted with C₁₋₆ alkyl, halo, hydroxyalkyl, hydroxy, alkoxy or keto;
R⁵ is hydrogen or C₁₋₆ haloalkyl;
R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with halo, C₁₋₆ alkyl, C₁₋₆ haloalkyl, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —N(R¹⁰)(R 1), or cyano;
D is C₁₋₃ alkyl, C₂₋₃ alkenyl, C₂₋₃ alkenyl, aryl, heteroaryl, C₃₋₈ cycloalkyl or heterocyclyl wherein said aryl, heteroaryl, cycloalkyl and heterocyclyl groups, which may be monocyclic or bicyclic, are optionally substituted on either the carbon or the heteroatom with one to five substituents selected from C₁₋₆ alkyl, halo or keto;
R⁷ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹⁰, —C(O)OSi[CH(CH₃)₂]₃, —OR¹⁰, —C(O)R¹⁰, —R¹⁰C(O)R⁹, —C(O)R⁹, —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², —SO(R¹²), —SO₂R⁹, —SO₂N(R^{c})(R^{d}), —SO₂CH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)(R⁹), R¹⁰S—, —C(R^{a})(R^{b})NR^{a}C(R^{a})(R^{b})(R⁹), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a}) or C(R^{a})(R^{b})C(O)N(R^{a})(R^{b}); wherein said alkyl, alkenyl, alkynyl, alkoxy, aryl, heteroaryl, cycloalkyl and heterocyclyl groups are optionally substituted on either the carbon or the heteroatom with one to five substituents independently selected from C₁₋₆ alkyl, halo, keto, cyano, haloalkyl, hydroxyalkyl, —OR⁹, —NO₂, —NH₂, —NHS(O)₂R⁸, —R⁹SO₂R¹², —SO₂R¹², —SO(R¹²), —SO₂N(R^{c})(R^{d}), —SO₂N(R¹⁰)C(O)(R¹²), —C(R¹⁰)(R¹¹)N(R¹⁰)(R¹¹), —C(R¹⁰)(R¹¹)OH, —COOH, —C(R^{a})(R^{b})C(O)N(R^{a})(R^{b}), —N(R¹⁰)C(R¹⁰)(R¹¹)(R⁹), —NH(CH₂)₂OH, —NHC(O)OR¹⁰, —Si(CH₃)₃, heterocycyl, aryl or heteroaryl;
R⁸ is hydrogen or C₁₋₆ alkyl;
or R⁴ and R⁸ or can be taken together with any of the atoms to which they may be attached or are between them to form a 4-10 membered heterocyclyl ring system wherein said ring system, which may be monocyclic or bicyclic, is optionally substituted with C₁₋₆ alkyl, halo, hydroxyalkyl, hydroxy, keto, —OR¹⁰, —SR¹⁰ or —N(R¹⁰)₂;
R⁹ is hydrogen, aryl, aryl(C₁₋₄) alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with halo or alkoxy;
R¹⁰ is hydrogen or C₁₋₆ alkyl;
R¹¹ is hydrogen or C₁₋₆ alkyl;
R¹² is hydrogen or C₁₋₆ alkyl which is optionally substituted with halo, alkoxy, cyano, —NR¹⁰ or —SR¹⁰;
R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl or heterocyclyl, wherein said alkyl, aryl, heteroaryl, C₃₋₈ cycloalkyl and heterocyclyl are optionally substituted on either the carbon or the heteroatom with C₁₋₆ alkyl or halo;
R^{b} is hydrogen, C₁₋₆ alkyl, (C₁₋₆ alkyl)aryl, (C₁₋₆ alkyl)hydroxyl, alkoxyl, hydroxyl, halo, aryl, heteroaryl, C₃₋₈ cycloalkyl or heterocyclyl, wherein said alkyl, aryl, heteroaryl, C₃₋₈ cycloalkyl and heterocyclyl are optionally substituted on either the carbon or the heteroatom with C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with C₁₋₆ alkyl and halo;
n is an integer from zero to three;
or a pharmaceutically acceptable salt, stereoisomer or N-oxide derivative thereof.

4. The method of claim 3 wherein R¹ is hydrogen; R² is hydrogen; or R¹ and R² can be taken together with the carbon atom to which they are attached to form a C₃₋₈ cycloalkyl ring wherein said cycloalkyl ring is optionally substituted with one or two substituents independently selected from C₁₋₆ alkyl, hydroxyalkyl, haloalkyl, aryl, heteroaryl, heterocyclyl or halo; or a pharmaceutically acceptable salt, stereoisomer or N-oxide derivative thereof.

5. The method of claim 4 wherein R³ is hydrogen; R⁴ is C₁₋₆ alkyl wherein said alkyl group is optionally substituted with C₃₋₆ cycloalkyl, aryl or halo; or a pharmaceutically acceptable salt, stereoisomer or N-oxide derivative thereof.

6. The method of claim 5 wherein R⁵ is hydrogen and R⁶ is C₁₋₆ haloalkyl; or a pharmaceutically acceptable salt, stereoisomer or N-oxide derivative thereof.

7. The method of claim 1 wherein the papain family cysteine protease inhibitor is selected from
N¹-(cyanomethyl)N²-(2,2,2-trifluoro-1-phenylethyl)-L-leucinamide;
N¹(cyanomethyl)-N²{(1S)-2,2,2-trifluoro-1-[4′-(methylsulfonyl)-1,1′-biphenyl-4-yl]ethyl}-L-leucinamide;
N¹-[(1S)-1-cyano-2-phenylethyl]-N²-[(1S)-1-(2′,6′-difluorobiphenyl-4-yl)-2,2-difluoroethyl]-L-leucinamide;
N¹-[(1R,2R)-1-cyano-2-phenylcyclopropyl]-N²-[(1S)-1-(2′,6′-difluorobiphenyl-4-yl)-2,2,2-trifluoroethyl]-L-leucinamide;
N¹-[(1S,2S)-1-cyano-2-phenylcyclopropyl]-N²-[(1S)-1-(2′,6′-difluorobiphenyl-4-yl)-2,2,2-trifluoroethyl]-L-leucinamide;
N¹-[(1S,2S)-1-cyano-2-phenylcyclopropyl]-N²-[(1S)-1-(2′,6′-difluorobiphenyl-4-yl)-2,2,2-trifluoroethyl]-L-leucinamide;
N¹-[(1S)-1-cyano-2-phenylethyl]-N²-{(1S)-2,2,2-trifluoro-1-[4′-(methylsulfonyl)biphenyl-4-yl]ethyl}-L-leucinamide;
N¹-[(1R)-1-cyano-2-(methylsulfonyl)ethyl]-N²-{(1S)-2,2,2-trifluoro-1-[4′-(methylsulfonyl)biphenyl-4-yl]ethyl}-L-leucinamide;
N¹-(1-cyanocyclopropyl)-N²-{(1S)-2,2,2-trifluoro-1-[4-(6-methylpyridin-2-yl)phenyl]ethyl}-L-leucinamide;
N¹-[(1S)-1-cyano-3-(methylthio)propyl]-N²-{(1S)-2,2,2-trifluoro-1-[4′-(methylsulfonyl)biphenyl-4-yl]ethyl}-L-leucinamide;
(2S)-4,4-dichloro-N-(1-cyanocyclopropyl)-2-({(1S)-2,2,2-trifluoro-1-[4′-(methylsulfonyl)biphenyl-4-yl]ethyl}amino)butanamide;
N¹-[(1S)-1-cyano-3-(methylsulfonyl)propyl]-N²-{(1S)-2,2,2-trifluoro-1-[4′-(methylsulfonyl)biphenyl-4-yl]ethyl}-L-leucinamide;
N¹-(1-cyanocyclopropyl)-N²-[(1S)-1-(4′-{1-[(cyclopropylamino)carbonyl]cyclopropyl} biphenyl-4-yl)-2,2,2-trifluoroethyl]-L-leucinamide;
N¹-(1-cyanocyclopropyl)-4-fluoro-N²-{(1S)-2,2,2-trifluoro-1-[4′-methoxy-3′-(methylsulfonyl)biphenyl-4-yl]ethyl}-L-leucinamide;
N¹-[cyano(phenyl)methyl]-N²-{(1S)-2,2,2-trifluoro-1-[4′-(methylsulfonyl)biphenyl-4-yl]ethyl}-L-leucinamide;
or a pharmaceutically acceptable salt, stereoisomer or N-oxide derivative thereof.

8. The method of claim 1 comprising another agent selected from the group consisting of nifurtimox, benznidazole, allopurinol, terbinafine, lovastatin, ketoconazole, itraconazole, posaconazole, miltefosine, ilmofosine, pamidronate, alendronate, risedronate, chloroquine, proguanil, mefloquine, quinine, pyrimethamine-sulphadoxine, doxocycline, berberine, halofantrine, primaquine, atovaquone, pyrimethamine-dapsone, artemisinin, quinhaosu. meglumine antimonite, sodium stibogluconate, amphotericin B, praziquantel, oxamniquine, pentamidine, melarsoprol, suramin and eflornithine.

9. A pharmaceutical composition comprising a papain family cysteine protease inhibitor of formula I: 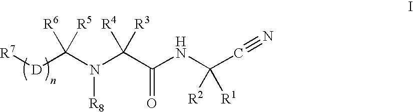
wherein R¹ is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
R² is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, C₃₋₆ cycloalkyl, halo, hydroxyalkyl, hydroxy, alkoxy or keto;
or R¹ and R² can be taken together with the carbon atom to which they are attached to form a C₃₋₈ cycloalkyl ring or heterocyclyl ring wherein said cycloalkyl and heterocycl rings are optionally substituted with one or two substituents independently selected from C₁₋₆ alkyl, hydroxyalkyl, haloalkyl, aryl, heteroaryl, heterocyclyl or halo;
R³ is hydrogen, C₁₋₆ alkyl or C₂₋₆ alkenyl wherein said alkyl and alkenyl groups are optionally substituted with C₃₋₆ cycloalkyl, aryl or halo;
R⁴ is hydrogen, C₁₋₆ alkyl or C₂₋₆ alkenyl wherein said alkyl and alkenyl groups are optionally substituted with C₃₋₆ cycloalkyl, aryl or halo;
or R³ and R⁴ can be taken together with the carbon atom to which they are attached to form a C₃₋₈ cycloalkyl ring, C₅₋₈ cycloalkenyl ring, or five to seven membered heterocyclyl ring wherein said cycloalkyl, cycloalkenyl and heterocyclyl rings are optionally substituted with C₁₋₆ alkyl, halo, hydroxyalkyl, hydroxy, alkoxy or keto;
R⁵ is hydrogen or C₁₋₆ haloalkyl;
R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with halo, C₁₋₆ alkyl, C₁₋₆ haloalkyl, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —N(R¹⁰)(R¹¹), or cyano;
D is C₁₋₃ alkyl, C₂₋₃ alkenyl, C₂₋₃ alkenyl, aryl, heteroaryl, C₃₋₈ cycloalkyl or heterocyclyl wherein said aryl, heteroaryl, cycloalkyl and heterocyclyl groups, which may be monocyclic or bicyclic, are optionally substituted on either the carbon or the heteroatom with one to five substituents selected from C₁₋₆ alkyl, halo or keto;
R⁷ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹⁰, —C(O)OSi[CH(CH₃)₂]₃, —OR¹⁰, —C(O)R¹⁰, —R¹⁰C(O)R⁹, —C(O)R⁹, —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², —SO(R¹²), —SO₂R⁹, —SO₂N(R^{c})(R^{d}), —SO₂CH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)(R⁹), R¹⁰S—, —C(R^{a})(R^{b})NR^{a}C(R^{a})(R^{b})(R⁹), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a}) or C(R^{a})(R^{b})C(O)N(R^{a})(R^{b}); wherein said alkyl, alkenyl, alkynyl, alkoxy, aryl, heteroaryl, cycloalkyl and heterocyclyl groups are optionally substituted on either the carbon or the heteroatom with one to five substituents independently selected from C₁₋₆ alkyl, halo, keto, cyano, haloalkyl, hydroxyalkyl, —OR⁹, —NO₂, —NH₂, —NHS(O)₂R⁸, —R⁹SO₂R¹², —SO₂R¹², —SO(R¹²), —SO₂N(R^{c})(R^{d}), —SO₂N(R¹⁰)C(O)(R¹²), —C(R¹⁰)(R¹¹)N(R¹⁰)(R¹¹), —C(R¹⁰)(R¹¹)OH, —COOH, —C(R^{a})(R^{b})C(O)N(R^{a})(R^{b}), —N(R¹⁰)C(R¹⁰)(R¹¹)(R⁹), —NH(CH₂)₂OH, —NHC(O)OR¹⁰, —Si(CH₃)₃, heterocycyl, aryl or heteroaryl;
R⁸ is hydrogen or C₁₋₆ alkyl;
or R⁴ and R⁸ or can be taken together with any of the atoms to which they may be attached or are between them to form a 410 membered heterocyclyl ring system wherein said ring system, which may be monocyclic or bicyclic, is optionally substituted with C₁₋₆ alkyl, halo, hydroxyalkyl, hydroxy, keto, —OR¹⁰, —SR¹⁰ or —N(R¹⁰)₂;
R⁹ is hydrogen, aryl, aryl(C₁₁₄) alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with halo or alkoxy;
R¹⁰ is hydrogen or C₁₋₆ alkyl;
R¹¹ is hydrogen or C₁₋₆ alkyl;
R¹² is hydrogen or C₁₋₆ alkyl which is optionally substituted with halo, alkoxy, cyano, —NR¹⁰ or —SR¹⁰;
R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl or heterocyclyl, wherein said alkyl, aryl, heteroaryl, C₃₋₈ cycloalkyl and heterocyclyl are optionally substituted on either the carbon or the heteroatom with C₁₋₆ alkyl or halo;
R^{b} is hydrogen, C₁₋₆ alkyl, (C₁₋₆ alkyl)aryl, (C₁₋₆ alkyl)hydroxyl, alkoxyl, hydroxyl, halo, aryl, heteroaryl, C₃₋₈ cycloalkyl or heterocyclyl, wherein said alkyl, aryl, heteroaryl, C₃₋₈ cycloalkyl and heterocyclyl are optionally substituted on either the carbon or the heteroatom with C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with C₁₋₆ alkyl and halo;
n is an integer from zero to three;
and another agent selected from the group consisting of nifurtimox, benznidazole, allopurinol, terbinafine, lovastatin, ketoconazole, itraconazole, posaconazole, miltefosine, ilmofosine, pamidronate, alendronate, risedronate, chloroquine, proguanil, mefloquine, quinine, pyrimethamine-sulphadoxine, doxocycline, berberine, halofantrine, primaquine, atovaquone, pyrimethamine-dapsone, artemisinin, quinhaosu. meglumine antimonite, sodium stibogluconate, amphotericin B, praziquantel, oxamniquine, pentamidine, melarsoprol, suramin and eflornithine.
